(19) United States
(12) Patent Application Publication
(10) Pub. No.: US 2007/0005066 A1
(43) Pub. Date: Jan. 4, 2007

(54) Modular nut plate

(76) Inventors: Lawrence W. Johnson, Taylor, MI (US); Edward J. Wochaski, Clinton Township, MI (US)

Correspondence Address: ILLINOIS TOOL WORKS INC., 3600 WEST LAKE AVENUE, PATENT DEPARTMENT, GLENVIEW, IL, 60025, US

(21) Appl. No.: 11/374,087

(22) Filed: Mar. 13, 2006

Related U.S. Application Data
(60) Provisional application No. 60/691,746, filed on Jun. 17, 2005.

(51) Int. Cl. A61F 2/30

(52) U.S. Cl. 606/69

(57) ABSTRACT

A modular nut plate includes a carrier configured for mounting the nut plate and fastener components selectably connectable to the carrier. Multiple nut plates can be linked together end to end.

### CROSS-REFERENCE TO RELATED APPLICATIONS

The present regular U.S. Patent Application claims the benefits of U.S. Provisional Application Ser. No. 60/671,746, filed on Jun. 17, 2005.

### FIELD OF THE INVENTION

The present invention relates generally to industrial fastener systems; and, more particularly, the invention pertains to so-called “nut plates” that provide a one or a plurality of fastener sites on a single device connected in an assembly.

### BACKGROUND OF THE INVENTION

Assemblies of various types such as, for example, automobiles, appliances and the like, use so-called “nut plates” as part of a fastener system. In many situations, it is difficult to access a nut, bolt head or other fastener component to complete an assembly. As a remedy to this difficulty, it is known to secure the nut, bolt or other component in a fixed position for engagement with the complimentary component of the fastener system. Affixing a single nut or bolt can be difficult, and positioning of it can be imprecise. It is known to provide a nut or threaded boss on an otherwise generally plate-like structure which can then be secured in place. It is known to secure such nut plates by welding or the like directly to the frame in some assemblies, such as an automobile frame, and by other types of fasteners including push-in fasteners in other, more lightweight assemblies. A single nut plate can be of sufficient size to include several fastener components.

It is necessary in some assemblies to use the same or similar nut components on nut plates of different size because the spacing between adjacent fasteners is different in different parts of the assembly. In known constructions for nut plates, this has required stocking and making available complete nut plates of different overall size, even though the hut components of the different nut plates are the same. Further, although the nut portion might be required to be of metal or other strong, durable material, the plate portion of a nut plate is needed primarily for positioning purposes and may not have the same requirements of strength and durability. However, if the nut plate is a monolithic body, the plate portion is of the same material, thus adding to cost of the nut plate.

Further, nut plates have been provided only in defined sizes with defined numbers of and locations for fastener sites. This again has added to both expense and inconvenience in that many different types of nut plates may be required.

What is needed is a modular nut plate adaptable to different uses.

### SUMMARY OF THE INVENTION

The present invention provides a modular nut plate having a carrier with a plurality of connector sites and one or more fastener component connected to the carrier at the connector sites.

In one aspect thereof, the present invention provides a modular nut plate with a carrier having first and second connector sites and a mounting structure for affixing the carrier in an assembly. A fastener component is selectably connected at one of the first and second connector sites. The fastener component has a fastener element thereon.

In another aspect thereof, the present invention provides a modular nut plate with a carrier having mounting structure for attaching the carrier in an assembly; and a fastener component having a fastener element. A snap connection first piece is provided on the carrier; and a snap connection second piece is provided on the fastener component. The first and second pieces are configured for connecting the fastener component to the carrier.

In a still further aspect thereof, the present invention provides a modular nut plate with a carrier having mounting structure configured for mounting the carrier to an assembly. The carrier includes a body having first and second connector sites configured as part of a snap together connection. A fastener component has a base, a fastener element on the base and at least a first connector end having a snap connection second part for selectably connecting to the first and second connector sites of the carrier.

An advantage of the present invention is providing a modular nut plate which can be manufactured efficiently and inexpensively.

Another advantage of the present invention is providing a modular nut plate having a carrier that can be used with different types and sizes of connector assemblies.

Still another advantage of the present invention is providing a modular nut plate that can be connected in end-to-end relationship of infinite lengths.

Other features and advantages of the invention will become apparent to those skilled in the art upon review of the following detailed description, claims and drawings in which like numerals are used to designate like features.

### BRIEF DESCRIPTION OF THE DRAWINGS

FIG. 1 is a perspective view of a modular nut plate in accordance with the present invention;

FIG. 2 is an exploded view of the modular nut plate shown in FIG. 1; and

FIG. 3 is a longitudinal cross-sectional view of the nut plate shown in FIG. 1, illustrating the nut plate installed in an assembly.

Before the embodiments of the invention are explained in detail, it is to be understood that the invention is not limited in its application to the details of construction and the arrangements of the components set forth in the following description or illustrated in the drawings. The invention is capable of other embodiments and of being practiced or being carried out in various ways. Also, it is understood that the phraseology and terminology used herein are for the purpose of description and should not be regarded as limiting. The use herein of “including”, “comprising” and variations thereof is meant to encompass the items listed thereafter and equivalents thereof, as well as additional items and equivalents thereof.

### DETAILED DESCRIPTION OF THE PREFERRED EMBODIMENT

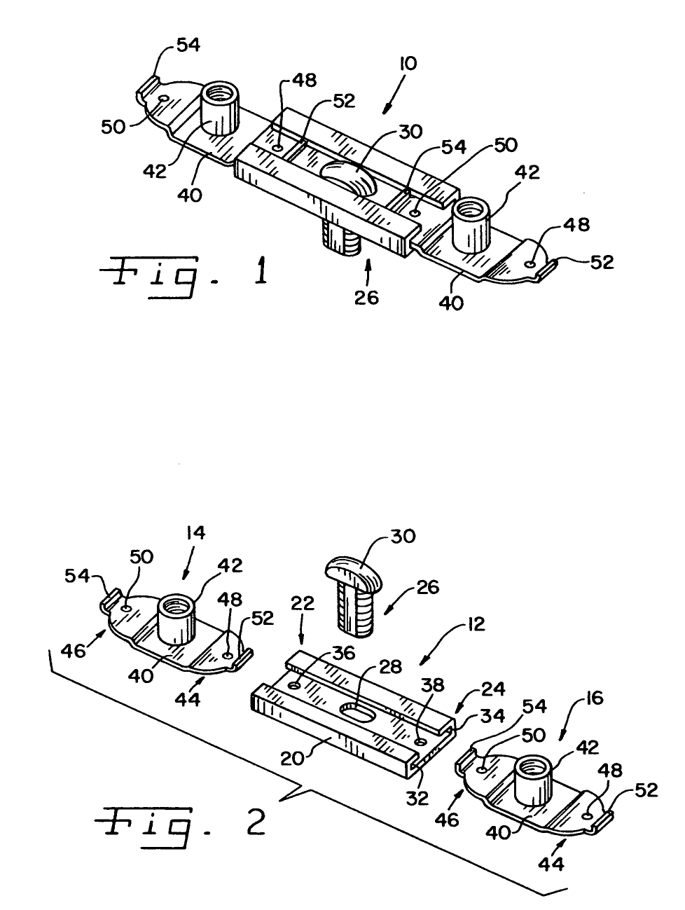

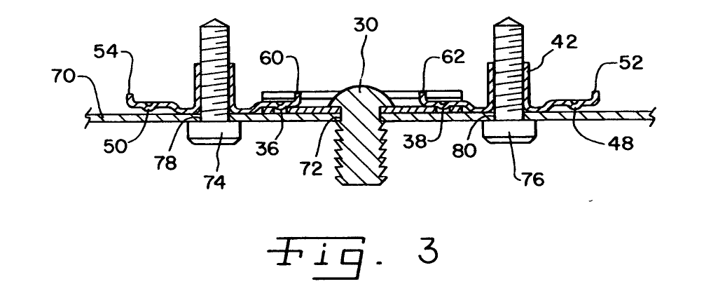

Referring now more specifically to the drawings and to FIG. 1 in particular, a modular nut plate 10 in accordance with the present invention is shown. Nut plate 10 includes a carrier 12, a first fastener component 14 connected at one end of carrier 12 and a second fastener component 16 connected at an opposite end of carrier 12 from the end at which fastener component 14 is connected. Since nut plate 10 is modular, the individual components thereof can be made of material most suitable for the requirements of the component. For example, carrier 12 can be made of plastic and fastener components 14 and 16 can be made of metal. Alternatively, one or both of fastener components 14 and 16 can be made of plastic and/or carrier 12 can be made of metal.

In the exemplary embodiment, carrier 12 is an elongate body 20 having firs and second connector sites 22 and 24 at opposite ends thereof. A mounting structure 26 includes an elongate hole 28 and a pin 30 for insertion and therethrough. In the exemplary embodiment, pin 30 is a so-called “Christmas Tree” fastener having a rectangular or flat, tapered body to prevent rotation when inserted in a rectangular hole. Other types of push-in fasteners, snap fasteners, threaded fasteners or the like can be used in place of pin 30. For long modular nut plates, more than a single fastener can be used.

Connector site 22 is configured for receiving and securing fastener component 14. Connector site 24 is configured for receiving and securing fastener component 16. In the exemplary embodiment, fastener component 14 and fastener component 16 are identical, and connector site 22 and connector site 24 are similarly structured for receiving connector components interchangeably.

Body 20 forms opposed, spaced, elongated channels 32, 34 along opposite sides thereof throughout the length of body 20. Channels 32 and 34 are part of connector site 22 and connector site 24. While each channel 32 and channel 34 is shown as a single continuous channel throughout the length of body 20, it should be understood that channels 32 and 34 can be shorter discontinuous channels provided at each first connector site 22 and second connector site 24. A depression or hole 36 is provided at connector site 22, and a depression or hole 38 is provided at connector site 24.

Fastener component 14 is a substantially symmetrical body having a base 40 and a fastener element 42. In the exemplary embodiment, fastener component 42 is an internally threaded boss. However, it should be understood that an externally threaded member or other type of fastener, either threaded or not threaded also can be supported by base 40. The present invention is not limited in its application to the use of internally threaded fastener elements, nor is the present invention limited to the use of threaded fastener elements in general.

Fastener component 14 further includes first and second connector ends 44 and 46 at opposite ends of base 40. Connector ends 44 and 46 include dimples 48 and 50, respectively. Connector ends 44 and 46 further define lips 52 and 54, respectively.

Second fastener component 16, is structured similarly to fastener component 14. Accordingly, the same reference numerals have been applied to fastener component 16, to designate the parts thereof similar to first fastener component 14.

First and second connector sites 22, 24 and first and second connector ends 44, 46 are configured as cooperating pieces of a snap together connection. Accordingly, first fastener component 14 and second fastener component 16 can be connected to carrier 12 without the use of tools.

In the assembly of modular nut plate 10, first fastener component 14 is attached to carrier 12 by inserting either connector end 44 or connector end 46 in to channels 32 and 34 and slid therein until dimple 48 or 50 engages hole 36. Registration of a dimple 48 or 50 in hole 36 establishes a fixed overall length for modular nut plate 10, and thereby the position of fastener element 42 relative to mounting structure 26. Thus, by accurately positioning the plate through installation via mounting structure 26, fastener element 42 is accurately and precisely located.

Second fastener component 16 is attached to carrier 12 at second connector site 24 in the same manner as attaching first fastener component 14 at first connector site 22.

To further establish fixed positioning of fastener components 14 and 16 relative to carrier 12, channels 32 and 34 can be provided with slots 60, 62 as illustrated in FIG. 3 for channel 34. Lips 52, 54 register in slots 60, 62 to further establish fixed positioning and secure location of fastener components 14 and 16 relative to carrier 12. Further, connector ends 44, 46 can be angled to define a longitudinal thickness between highest and lowest portions thereof greater than a thickness of channels 32, 34. Thus, an interference fit is established between fastener component 14 or 16 and carrier 12.

Modular nut plate 10 is connected in an assembly 70 having an elongated hole 72 for receiving fastener 30. Thereafter, bolts 74, 76 can be inserted into fastener elements 42 via holes 78, 80 in assembly 70.

While shown herein with similar first and second components 14, 16 a modular nut plate 10 in accordance with the present invention can be assembled with different fastener components on opposite ends of carrier 12. Further, since fastener components 14, 16 are symmetrical, with connector ends on opposite longitudinal ends thereof, it should be understood by those skilled in the art that additional carriers 12 and fastener components 14, 16 can be connected end for hand in any number or length.

Variations and modifications of the foregoing are within the scope of the present invention. It is understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention. The claims are to be construed to include alternative embodiments to the extent permitted by the prior art.

Various features of the invention are set forth in the following claims.

What is claimed is:

1. A modular nut plate comprising:
a carrier having first and second connector sites and a mounting structure for affixing said carrier in an assembly; and
a fastener component selectably connected at one of said first and second connector sites, said fastener component having a fastener element thereon.

2. The modular nut plate of claim 1, said fastener element being a threaded boss.

3. The modular nut plate of claim 1, including a second fastener component selectably connected to the other of said first and second connector sites.

4. The modular nut plate of claim 1, said fastener component having first and second connector ends, each said connector end being selectably connectable to said first and second connector sites on said carrier.

5. The modular nut plate of claim 4, said connector sites and said connector ends comprising cooperating parts of a snap-together connection.

6. The modular nut plate of claim 5, said fastener elements being threaded bosses.

7. The modular nut plate of claim 1, said connector sites and said connector ends comprising cooperating parts of a snap-together connection.

8. A modular nut plate comprising:
a carrier having mounting structure for attaching said carrier in an assembly;
a fastener component having a fastener element;
a snap connection first piece on said carrier;
a snap connection second piece on said fastener component; and
said first and second pieces configured for connecting said fastener component to said carrier.

9. The modular nut plate of claim 8, said fastener element being a threaded boss.

10. The modular nut plate of claim 8, said carrier having two said snap connection first pieces at which said fastener component can be connected.

11. The modular nut plate of claim 8, said fastener component having two said snap connector second pieces connectable to said snap connector first piece.

12. The modular nut plate of claim 8, said carrier having an elongated body and a said snap connector first piece at each end thereof.

13. The modular nut plate of claim 12, having two said fastener components, one said fastener component connected to each said snap connector first piece.

14. A modular nut plate comprising:
a carrier having mounting structure configured for mounting said carrier to an assembly, said carrier including a body having first and second connector sites configured as part of a snap together connection; and
a fastener component having a base, a fastener element on said base and at least a first connector end having a snap connection part for selectably connecting to said first and second connector sites of said carrier.

15. The modular nut plate of claim 14, said fastener component having first and second connector ends each having a snap connection part for selectably connecting to said first and second connector sites of said carrier.

16. The modular nut plate of claim 14, said first and second connector sites including opposed spaced channels.

17. The modular nut plate of claim 16, each said first and second connector site including at least one of a dimple and a hole between said channels.

18. The modular nut plate of claim 17, said first connector end including at least one of a dimple and a hole for engagement with said one of a dimple and a hole at said first and second connector sites.

19. The modular nut plate of claim 14, said first and second connector sites each defining a slot and said connector end having a lip received in said slot.

20. The modular nut plate of claim 14, including a second fastener component having a base, a fastener element on said base and at least a first connector end having a snap connection part for selectably connecting to said first and second connector sites of said carrier.
